(19) United States
(12) Patent Application Publication
(10) Pub. No.: US 2026/0224311 A1
(43) Pub. Date: Aug. 6, 2026

(54) GUIDING A ROBOTIC SURGICAL SYSTEM TO PERFORM A SURGICAL PROCEDURE

(71) Applicant: IX Innovation LLC, Seattle, WA (US)

(72) Inventors: Justin Esterberg, Mercer Island, WA (US); Jeffrey Roh, Seattle, WA (US)

(21) Appl. No.: 19/631,113

(22) Filed: Mar. 27, 2026

Related U.S. Application Data
(63) Continuation-in-part of application No. 19/287,985, filed on Aug. 1, 2025.
Application No. 19/287,985 is a continuation of application No. 18/174,287, filed on Feb. 24, 2023, now abandoned.
Application No. 18/174,287 is a continuation of application No. 16/023,014, filed on Jun. 29, 2018, now Pat. No. 11,589,933.
(60) Provisional application No. 62/526,439, filed on Jun. 29, 2017.
(60) Provisional application No. 62/528,102, filed on Jul. 2, 2017.
(60) Provisional application No. 62/529,019, filed on Jul. 6, 2017.

(51) Int. Cl. A61B 34/30; A61B 17/00; A61B 17/16; A61B 34/00; A61B 34/10; A61B 34/20; A61B 90/00; A61B 90/50

(52) U.S. Cl. CPC A61B 34/30; A61B 17/1615; A61B 34/20; A61B 90/361; A61B 90/37; A61B 17/00234; A61B 17/1622; A61B 2034/102; A61B 2034/105; A61B 2034/107; A61B 2034/2048; A61B 2034/256; A61B 2034/305; A61B 34/74; A61B 2090/365; A61B 2090/371; A61B 2090/372; A61B 2090/3937; A61B 2090/502

(57) ABSTRACT

A robotic surgical system may be used to perform a surgical procedure. Providing guidance for the robotic surgical system includes integrating a Point of View (PoV) surgical drill with a camera to capture a PoV image of a surgical area of a subject patient; displaying an image of the surgical area, based on a viewing angle of the PoV surgical drill, thus enabling the surgeon to operate on the surgical area using the PoV surgical drill. The PoV surgical drill operates based on the surgeon's control of a guidance drill. The content of the images may change based on a change in the viewing angle of the PoV surgical drill.

### FIELD OF THE DISCLOSURE

The present disclosure is generally related to a robotic surgical system in a Virtual Reality (VR) environment. More particularly, the present disclosure is related to allowing a surgeon to control an endoscope during a surgical procedure.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Robotic surgical devices are now routinely used for numerous surgical procedures such as general surgery, pediatric surgery, and those related to the medical fields of gynecology, urology, cardiology, and otorhinolaryngology. Robotic devices continue to evolve and are being more frequently utilized in surgical procedures.

The robotic devices are used most in surgical procedures that require a high degree of accuracy and/or precision. Such robotic devices include autonomous, tele-operated, and interactive type robotic systems. Interactive robotic systems are most frequently used for providing the surgeon with direct hands-on control of the surgical procedure, thus achieving a high degree of accuracy and/or precision. For example, in a knee surgery, a surgeon can use an interactive robotic arm to sculpt a bone and/or to receive a knee implant. In a laparoscopic surgical procedure, using a robotic system, a surgeon may directly control and manipulate tissue, albeit at some distance from the patient through a fulcrum point in the abdominal wall.

In other surgical procedures performed using robotic devices, the surgeon may sit at a console in the operating room, but outside the sterile field, directing and controlling the movements of one or more robotic arms. However, robotic devices can be intrusive during a surgical procedure, blocking the surgeon's point of view and occupying substantial space around an operating table, increasing the likelihood of an operator error.

Instruments for robotic devices, like movement detection equipment and navigation markers, may be implemented in surgical procedures as safeguards. Such instruments help guide the robotic devices and assist the surgeons in avoiding errors. The movement detection equipment and the navigation markers help determine the position of the instrument in space and prevent the instrument from deviating beyond the path set by the surgeon. For example, in neurosurgery, neuromonitors with sensors are used to detect the threshold level, and a signal is sent to an appropriate system to stop insertion of the surgical instrument or to move the instrument away for preventing any damage when an error is detected as being imminent. Further, actuators are also used for controlled movement and positioning of end effectors in the robotic arm.

Endoscopy is a surgical procedure involving usage of endoscopes. The endoscopes are used for viewing and operating on internal body organs of a patient. Endoscopy allows the surgeons to view medical problems within a body of the patient by inserting an endoscope through a small incision, or mouth. The endoscope is a flexible tube comprising a camera for allowing the surgeons to examine the internal body organs of the patient. However, the use of such endoscopes for performing complex endoscopic surgeries such as a stomach surgery or a gallbladder surgery, may be challenging, and may cause discomfort to the patient.

Currently, in order to reduce the discomfort, the endoscopes may provide capabilities such as zoom-in, and zoom-out capabilities that may result in reducing a need to physically move the camera. Also, the endoscopes may provide auto-focus capabilities for capturing better images of the internal body organs of the patient. However, the endoscopes are heavy and large in size and are hard to handle by the surgeons. Further, while performing surgical procedures, the surgeons are generally unaware of a location of the endoscope lying within the body of the patient. Thus, such situations may result in adverse events such as perforating the internal body organs and internal vessels of the patient, during the surgical procedure.

Therefore, in order to track the location of the endoscopes and to reduce the adverse events, the endoscopes are integrated with tracking systems and software. The software retrieves data along with a video from the endoscopes, during the surgical procedure. For example, in an endonasal surgery, the software tracks the data along with a video from the endoscope. Further, the current tracking system provides a Graphical User Interface (GUI) for allowing the surgeons to start/stop recording, set mode, and display a progress of the recording. However, current tracking systems suffer from many shortcomings, such as not providing a “point of view” image, incapability to measure critical distances to the internal organs, and inability to interact with the surgeons during the surgical procedure. Therefore, there is a need for an improved system that may be efficient for performing the endoscopic surgeries.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 shows a network connection diagram 100 for a system 102 to guide a robotic surgical system when performing a surgical procedure, according to an embodiment.

FIG. 2 shows a virtual grid used to determine locations of different instruments present in a Three-Dimensional (3D) space corresponding to an operating table, according to an embodiment.

FIG. 3A shows a front point of view (PoV) of a surgical drill, according to an embodiment.

FIG. 3B shows a cross-section PoV of a surgical drill along a Z-Z′ axis, according to an embodiment.

FIG. 4 shows a Virtual Reality (VR) drill set at a first angle, relative to images of the subject patient, according to an embodiment.

FIG. 5 shows the VR drill set at a second angle relative to the images of the subject patient, according to an embodiment.

FIG. 6 illustrates a network connection diagram 600 of a system 602 for allowing a surgeon to control an endoscope 614 during a surgical procedure, according to an embodiment.

FIG. 7 illustrates a block diagram showing different components of the system 602, according to an embodiment.

FIG. 8 illustrates a flowchart 800 showing a method performed by a measurement recognition module 810 of the system 602, according to an embodiment.

FIGS. 9A and 9B collectively illustrate a Graphical User Interface (GUI) 900 of a user device 608 connected to the system 602, presenting information related to the surgical procedure, according to an embodiment.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

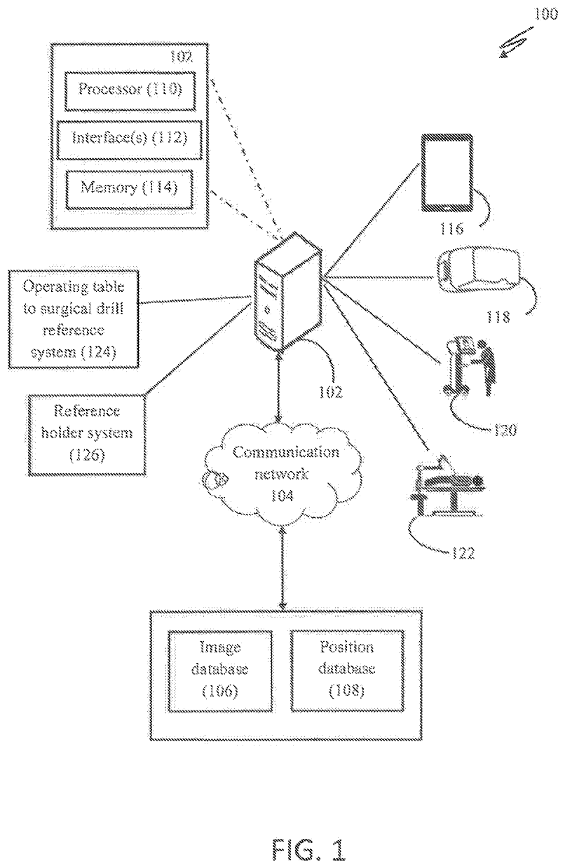

FIG. 1 shows a network connection diagram 100 for a system 102 to guide a robotic surgical system when performing surgery, according to an embodiment. The system 102 may be connected to a communication network 104. The communication network 104 may further be connected to an image database 106 and a position database 108 to facilitate data transfer therebetween with the system 102.

The image database 106 may store images of a subject patient, as well as images of previous patients who have undergone similar surgeries. The images may be captured using an X-ray, ultrasound, and Magnetic Resonance Imaging (MRI). Further, the images may be present in raw form, as Three-Dimensional (3D) models, Augmented Reality (AR) images, Virtual Reality (VR) images, and Point of View (PoV) images. The position database 108 may store real-time position information of a PoV surgical drill 122 and that of a virtual drill that may he shown to a surgeon during surgery.

The communication network 104 may be either of a wired and/or a wireless network. The communication network 104, if wireless, may be implemented using communication techniques such as Visible Light Communication (VLC), Worldwide Interoperability for Microwave Access (WIMAX), Long Term Evolution (LTE™), Wireless Local Area Network (WLAN), Infrared (IR) communication, Public Switched Telephone Network (PSTN), Radio waves, and other communication techniques known in the art.

The system 102 may further include a processor 110, interface(s) 112, and a memory 114. The processor 110 may execute an algorithm stored in the memory 114 for processing the PoV images and for guiding the robotic surgical system when performing a surgical procedure. The processor 110 may also be configured to decode and execute any instructions received from one or more other electronic devices or server(s).

In at least one embodiment, the processor 110 may include one or more general purpose processors (e.g., INTEL® or Advanced Micro Devices® (AMD) microprocessors) and/or one or more special purpose processors (e.g., digital signal processors or Xilinx System On Chip (SOC) Field Programmable Gate Array (FPGA) processor). The processor 110 may be configured to execute one or more computer-readable program instructions, such as program instructions to carry out any of the functions described in this description.

The interface(s) 112 may facilitate interaction between a surgeon and the system 102. The interface(s) 112 may accept an input from the surgeon or other user who is associated with an on-going surgery and/or provide an output to the surgeon or other user. The interface(s) 112 may either be a Command Line Interface (CLI), Graphical User Interface (GUI), or a voice interface.

The memory 114 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMS), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions.

FIG. 1 also shows a user device 116, AR/VR display 118, guidance drill 120, and a Point of View (PoV) surgical drill 122 that are all connected to the communication network 104. The surgeon may maneuver the guidance drill 120 to remotely control the actual PoV surgical drill 122. Locations and motions of the guidance drill 120 may be tracked at all times by an operating table to surgical drill reference system (hereafter “reference system”) 124 and a reference holder system 126. The reference holder system 126 and the reference system 124 may control the PoV surgical drill 122 based on the tracked locations and motions thereof.

The user device 116 is shown as a tablet in FIG. 1; however, other user devices having Graphical User Interfaces (GUIs) may also be used. Other implementations of user device 116 may include, but not be limited to, a smart phone, phablet, laptop, and desktop.

FIG. 1 also shows AR/VR display 118 as a VR glass in present case, although the example embodiments are not so limited.

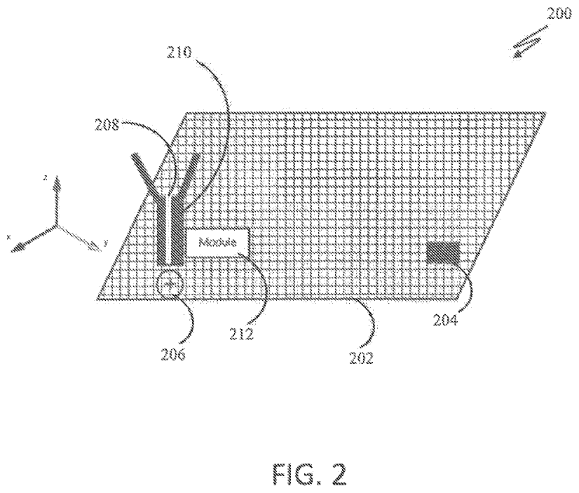

FIG. 2 shows a virtual grid 200 used to determine locations of different instruments present in a Three-Dimensional (3D) space, by the reference system 124, according to an embodiment. The virtual grid 200 is shown in relation to an operating table 202. A marker 204 for AR reference is used on the virtual grid 200 to help an AR imaging system determine a location of the AR reference. Based on identified location of the AR marker 204, the AR imaging system may identify a location of a drill bit reference 206. Using the location of the drill hit reference 206, the AR/VR display 118 may identify a location of a virtual drill in the 3D space. The virtual drill may be shown to the surgeon, using the AR/VR display 118, during an actual surgical procedure.

In at least one embodiment, a drill bit may be placed in an opening 208 of a drill holder 210 of the PoV surgical drill 122. Once the drill bit is placed in the drill holder 210, a module 212 connected to the drill holder 210 may identify parameters of PoV surgical drill 122, including a type of the surgical drill, a type of the drill bit, a size of the drill bit, and an absolute position of a tip of the drill bit in an XYZ coordinate system referencing the operating table 202.

As stated above, robotic devices include autonomous, tele-operated, and interactive type robotic systems. Interactive robotic systems are most frequently used for providing the surgeon with direct hands-on control of the surgical procedure, thus achieving a high degree of accuracy and/or precision. For example, in a knee surgery, a surgeon can use an interactive robotic arm to sculpt a bone and/or to receive a knee implant. In a laparoscopic surgical procedure, using a robotic system, a surgeon may directly control and manipulate tissue, albeit at some distance from the patient through a fulcrum point in the abdominal wall.

Accordingly, some non-limiting example embodiments may include drill bits for cutting soft tissue, e.g., with serrated edges, sheared angles, or other designs to improve cutting performance. Various embodiments of the drill bits have teeth disposed on an inner-housing, with an opening to direct a cutting surface away from the tissue to thereby provide a polished edge to the tissue.

Some embodiments of such drill bits may include serrated edges on internal and external cutting surfaces for aggressive resection of tissue; other drill bit embodiments may include internal serrated edges and a polished cutting surface, as described above, to leave a polished edge to the tissue; and still other drill bit embodiments may be non-serrated.

In at least one embodiment, the module 212 may further comprise a surgical drill reader configured to read a serial number present on the drill bit. The serial number may be related to the PoV surgical drill 122 and/or the drill bit thereof. Serial numbers respectively corresponding to different drill bits and different categories of surgical drills may be stored in a memory corresponding to the module 212. The received serial number may be matched with the serial numbers stored in the memory to identify details related to the PoV surgical drill 122 and the drill bit. In at least one example, the surgical drill reader may be implemented as a Near Field Communication (NFC) reader, and NFC encoded chip may be attached to the drill bit. The NFC reader may therefore communicate with the NFC encoded chip to receive the serial number of the drill bit.

In at least one embodiment, the module 212 may identify and/or determine the drill bit being cradled, reference the position of the drill bit with the virtual grid 200, identify the surgical drill and the drill bit, convert the surgical drill identification to an associated virtual surgical icon, and convert the drill bit identification to an associated virtual surgical drill bit icon. The module 212 may further transmit the virtual surgical icon and the virtual drill bit icon, referenced to the XYZ coordinate system of the operating table 202, to an AR imaging system and to the reference holder system 126.

In at least one other embodiment, the reference holder system 126, shown and described with regard to FIG. 1, may be an integral unit of the PoV surgical drill 122, configured to identify and store, in real-time, XYZ coordinates of the PoV surgical drill 122 and drill bit tip, and an angle of the PoV surgical drill 122. The reference holder system 126 may include an accelerometer to detect changes in position of the PoV surgical drill 122 e.g., a three-axis accelerometer. In at least one other example, the data recorded by the reference holder system 126 may be transmitted to the AR imaging system. The data may include the virtual icon of the PoV surgical drill 122 and the virtual icon of the drill bit along with their real positions relative to the operating table 202.

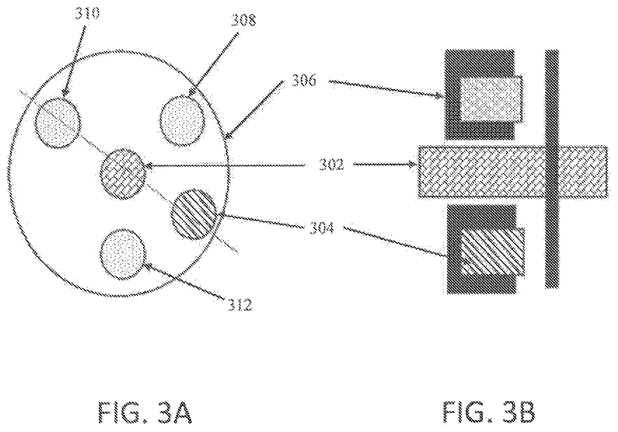

FIG. 3A shows a front view of the PoV surgical drill 122; and FIG. 3B shows a cross-section of the PoV surgical drill 122 along a Z-Z′ axis, in accordance with at least one example embodiment. Drill bit 302 is centrally located, length-wise, on PoV surgical drill 122. A Light Amplification by Stimulated Emission of Radiation (LASER) device 304 is disposed on the periphery of block 306 surrounding the drill bit 302. The LASER device 304 may guide a surgeon in a direction in which the PoV surgical drill 122 is moving. Further, cameras 308, 310, and 312 are shown on the periphery of the block surrounding the drill bit 302, although the cameras are not so limited in quantity. At least one of the cameras 308, 310, and 312 may capture a PoV image of a surgical area of a subject patient.

In at least one embodiment, images captured by any one or more of the cameras 308, 310, and 312 may be integrated to produce one composite PoV image using known image processing tools and techniques. In at least one example, the composite PoV image may be cropped in a circle and centered with regard to the drill bit 302 based on defaults settings stored by the surgeon. The cropped image may then be sent to the reference holder system 126.

In at least one embodiment, while performing a surgical procedure on the subject patient the surgeon may maneuver the guidance drill 120 to control the PoV surgical drill 122 based on the PoV images seen on the AR/VR display 118. As set forth above, the PoV images may be collected using one or more of the cameras 308, 310, and 312 positioned on the head of the PoV surgical drill 122. Thus, content of the PoV images may change based on an orientation and direction faced by the PoV surgical drill 122.

In at least one embodiment, the processor 110 may synchronize the position of the PoV surgical drill 122 with a reference linked with augmented images shown on the AR display device 118. Based on such synchronization, a Virtual Reality (VR) drill may be shown to the surgeon on the augmented images displayed using the AR display device 118. The VR drill may move based on changes in position of the PoV surgical drill 122, controlled by the surgeon controlling the guidance drill 120. Thus, such synchronization of the VR drill and the PoV surgical drill 122 provides a realistic experience to the surgeon. Further, operating room cameras may also be used for capturing images of the surgical procedure from a fixed angle, as set by the surgeon or based on a positioning of the operating room cameras. Such images may be stored in the image database 106, and may be displayed to the surgeon using an image display, e.g., AR display device 118.

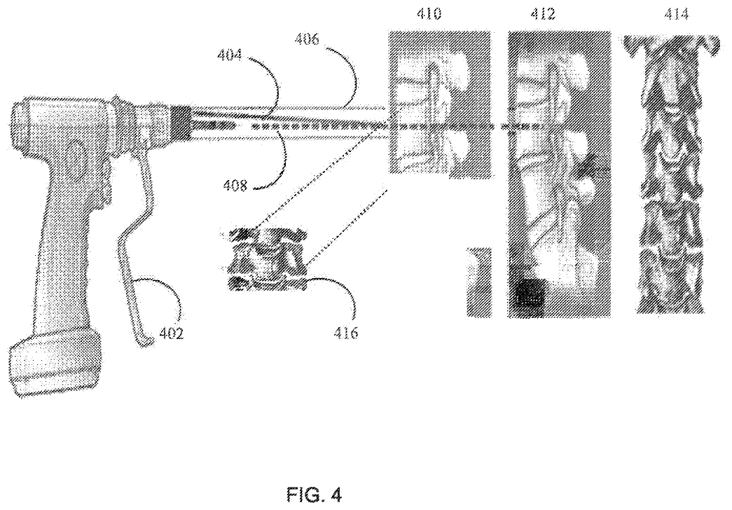

FIG. 4 shows the VR drill 402 set at a first angle, relative to images of the subject patient, according to an embodiment. The VR drill 402 is displayed, via a GUI, to the surgeon and other users associated with the surgery; and the VR drill 402 replicates the position and the direction of the PoV surgical drill 122. FIG. 4 also shows a LASER marker 404, a PoV image region 406, and a line of sight 408 of the VR drill 402 (replicating a line of sight of the PoV surgical drill 122). The surgeon may be able to see an actual image 410 of the, e.g., spine of the patient, an AR image 412 of the spine, and a VR image 414 of the spine. The images may be seen based on the surgeon's preferences e.g., images may be overlaid over each other, the images may be shown in parallel in a side to side arrangement, etc. A highlighted section 116 is also shown as the PoV image captured by one or more of the cameras 308, 310, and 312 of the PoV surgical drill 122, in accordance with at least one example.

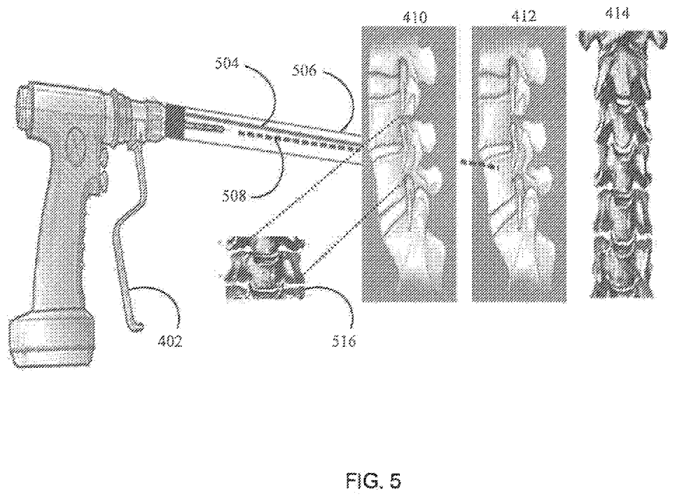

FIG. 5 shows the VR drill 402 set at a second angle relative to the images of the subject patient, according to an embodiment. FIG. 5 also shows a LASER marker 504, a PoV image region 506, and a line of sight 508 of the VR drill 402 (replicating a line of sight of the PoV surgical drill 122). The surgeon or other user associated with the surgery may be able to see the actual image 410 of, e.g., the spine of the patient, AR image 412 of the spine, and the VR image 414 of the spine. The images may be seen based on the surgeon's preferences. A highlighted section 516 is also shown as the PoV image captured by at least one of the cameras 308, 310, and 312 of the PoV surgical drill 122, in at accordance with least one example. As evident from comparison of the FIG. 4 and FIG. 5, the position and the direction of the VR drill has changed and thus, content of the PoV images 416 and 516 captured by the cameras of the PoV surgical drill is different. In this way, the surgeon may leverage different points of view to improve accuracy and to reduce errors while performing the surgical procedure.

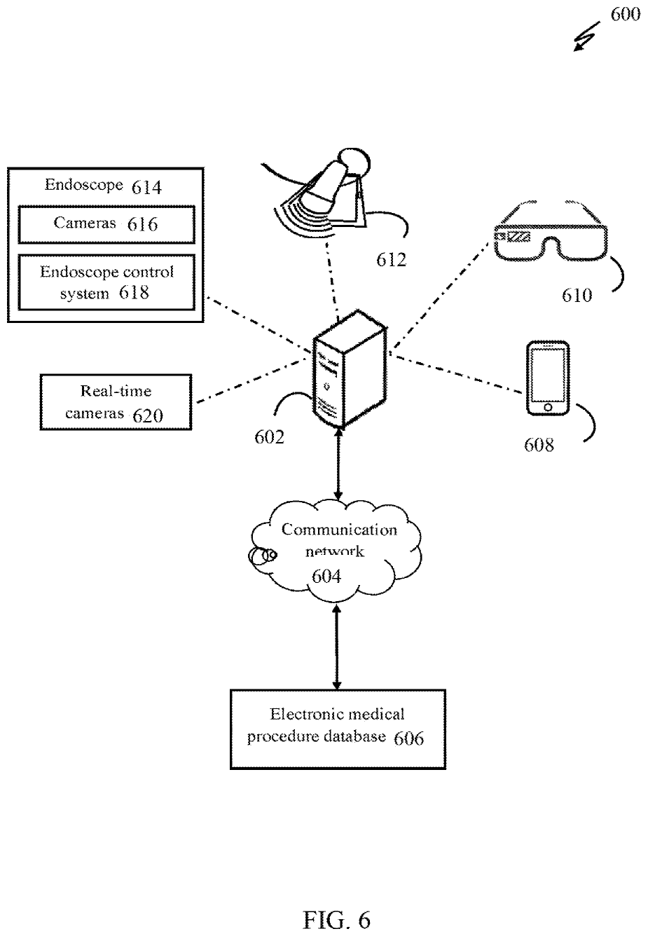

FIG. 6 illustrates another example embodiment of a network connection diagram 600 of a system 602 for allowing a surgeon to control an endoscope 614 during a surgical procedure. The system 602 may be connected to a communication network 604. The communication network 604 may further be connected with an electronic medical procedure database 606 for allowing data transfer between the system 602 and the electronic medical procedure database 606.

The communication network 604 may be a wired and/or a wireless network. The communication network 604, if wireless, may be implemented using communication techniques such as Visible Light Communication (VLC), Worldwide Interoperability for Microwave Access (WiMAX), Long Term Evolution (LTE™), Wireless Local Area Network (WLAN), Infrared (IR) communication, Public Switched Telephone Network (PSTN), Radio waves, and other communication techniques known in the art.

The electronic medical procedure database 606 may include different information required during the surgical procedure. A single database is used in the in present case, however different databases may also be used for storing the data. The electronic medical procedure database 606 may be configured to store data of patients in a real-time. The data may correspond to medical imaging data, and/or a diagnostic data. Examples of the data may include medical records of the patients, such as previous medical history of the patients, medical charts, test results, and notes of surgeons/doctors or medicine providers.

In one embodiment, the electronic medical procedure database 606 may be configured to store recording of the surgical procedure in a real-time. The electronic medical procedure database 606 may be configured to store data such as video data and camera data obtained during an execution of the surgical procedure. Further, the electronic medical procedure database 606 may be configured to store results of previous surgeries performed on previous patients. The results of previous surgeries may be stored in a structured manner. The electronic medical procedure database 606 may further store images of the patients. The images may be any of camera images, Magnetic Resonance Imaging (MRI) images, and X-Ray images. The electronic medical procedure database 606 may also comprise unexpected or adverse events occurring in a time-sequence of actual results. Also, the electronic medical procedure database 606 may be configured to store Augmented Reality (AR) images. In one case, for a particular surgical procedure performed on a particular patient, the electronic medical procedure database 606 may store information related to each step performed during the surgical procedure.

In one embodiment, the electronic medical procedure database 606 may be configured to store inputs that may be provided by the surgeon during the surgical procedure. It should be noted that the surgeon may provide the inputs either using the system 602 or a user device 608. A smart phone is shown as the user device 608 in FIG. 6, as an example. Further, the user device 608 used for displaying information related to the surgical procedure may be any other device comprising a Graphical User Interface (GUI), for example, a laptop, a desktop, a tablet, a phablet, or other such devices known in the art.

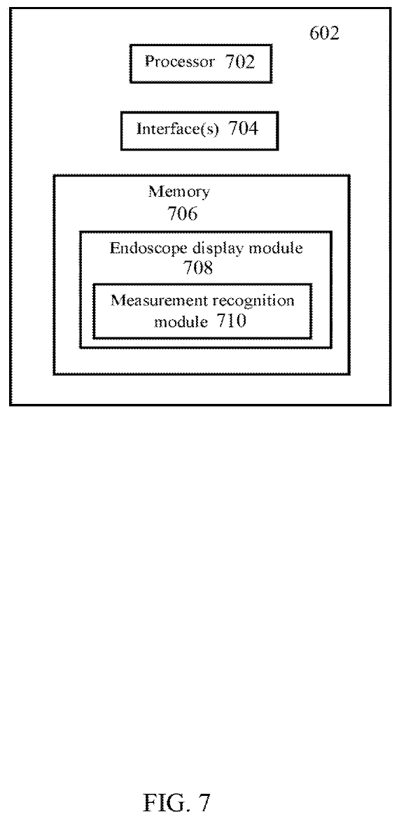

In one embodiment, referring to FIG. 7, a block diagram showing different components of the system 602 is explained. The system 602 includes a processor 702, interface(s) 704, and a memory 706. The processor 702 may execute an algorithm stored in the memory 706 for allowing the surgeon to control the endoscope 614 during the surgical procedure. The processor 702 may also be configured to decode and execute any instructions received from one or more other electronic devices or server(s). The processor 702 may include one or more general purpose processors (e.g., INTEL® or Advanced Micro Devices® (AMD) microprocessors) and/or one or more special purpose processors (e.g., digital signal processors or Xilinx® System On Chip (SOC) Field Programmable Gate Array (FPGA) processor). The processor 702 may be configured to execute one or more computer-readable program instructions, such as program instructions to carry out any of the functions described in this description.

The interface(s) 704 may help the surgeon to interact with the system 602. The interface(s) 704 of the system 602 may either accept an input from the surgeon or provide an output to the surgeon, or may perform both the actions. The interface(s) 704 may either be a Command Line Interface (CLI), Graphical User Interface (GUI), or a voice interface.

The memory 706 may include, but is not limited to, fixed (hard) drives, magnetic tape, floppy diskettes, optical disks, Compact Disc Read-Only Memories (CD-ROMs), and magnetooptical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions.

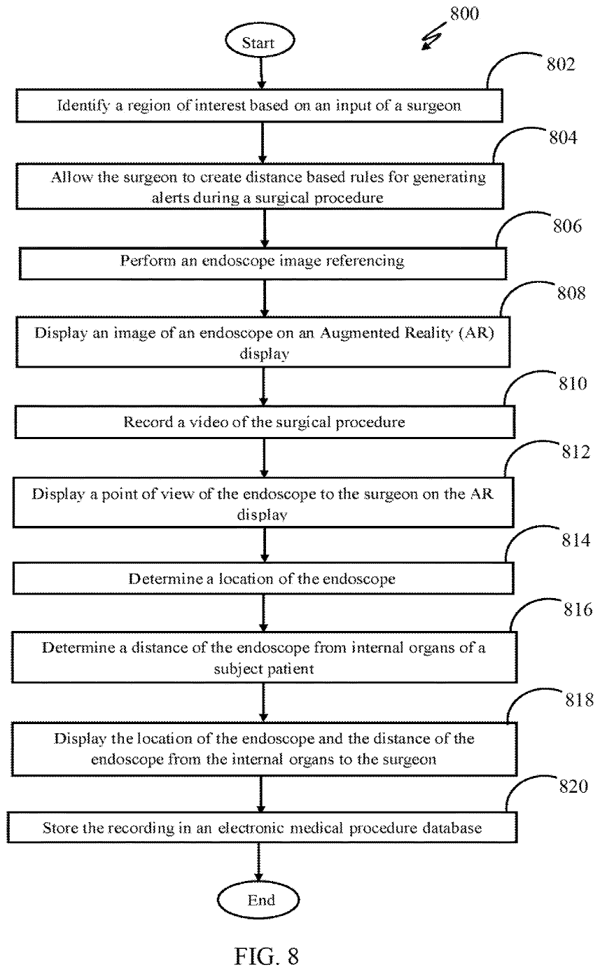

In one embodiment, the memory 706 may include an endoscope display module 708. The endoscope display module 708 may include a measurement recognition module 710. Functioning of the measurement recognition module 710 will now be explained with reference to flowchart 800, shown in FIG. 8. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

At first, the surgeon may need to log-in to the system 602. The surgeon may log-in using his credentials i.e. a user name and a password, in one case. For example, in a scenario, the surgeon may log-in using the user device 608. Upon log-in, the measurement recognition module 710 may identify a region of interest based on an input of the surgeon, at step 802. The region of interest may correspond to an affected body part of a subject patient. The subject patient may refer to a patient that needs to be operated. In one case, the input of the surgeon, for identifying the region of interest, may be determined either in reference to real images or Augmented Reality (AR) images of the subject patient. The real images may be captured using real-time cameras 620 and the AR images may be captured using Magnetic Resonance Imaging (MRI). Thereafter, the surgeon may identify a problem or an abnormal growth region within a body of the subject patient. For example, in an image of the subject patient, captured using MRI, an abnormal growth region may be identified in a right lung. Such abnormal growth region may correspond to the region of interest.

Successive to identifying the region of interest at step 802, the measurement recognition module 710 may allow the surgeon to create distance based rules, at step 804. The distance based rules may be used for generating alerts during the surgical procedure. The distance based rules may be created based on the AR images, the real images, and an image of the endoscope 614. The distance based rules may include a distance between one or more internal organs of the subject patient, distance between an image of the endoscope 614 and the one or more internal organs, and critical distances for generating alarms. In one case, an alarm may be generated while the endoscope 614 is present within a distance of ‘10 mm’ from an internal organ.

Post creating the distance based rules, the measurement recognition module 710 may perform an endoscope image referencing, at step 806. In the endoscope image referencing, the images of the subject patient captured by the real-time cameras 620 may be overlaid with the AR images, such that the surgeon may view the real images of the subject patient and the AR images. Thereafter, the surgeon may start performing the surgical procedure on the subject patient by inserting the endoscope 614 within the body of the subject patient. Thereafter, the measurement recognition module 710 may allow the surgeon to control the endoscope 614 using an endoscope control system 618.

Successive to performing the endoscope image referencing, the measurement recognition module 710 may display the image of the endoscope 614 on an AR display 610, at step 808. For example, the AR display 610 may correspond to an AR glass, worn by the surgeon. In one case, the image of the endoscope 614 may be provided by an ultrasound device 612.

Thereafter, the measurement recognition module 710 may initiate recording a video of the surgical procedure, at step 810. In one case, the video may be recorded using cameras 616 positioned on a head of the endoscope 614. Thereafter, the measurement recognition module 710 may display a point of view of the endoscope 614, to the surgeon using the AR display 610, at step 812. The point of view may be captured by the cameras 616 of the endoscope 614. For example, a “sub window” in the AR display 610 may display the cameras 616 of the endoscope 614. In another example, a “window” may automatically change its size based upon the distance between the endoscope 614 and the one or more internal organs. For instance, when the endoscope 614 is very far away from the one or more internal organs i.e., at 10 mm distance, then the window may zoom-out to fill the AR display 610. On the other hand, when the endoscope 614 is too close to the one or more internal organs, for instance at 5 mm, then the window may zoom-in.

In one embodiment, the measurement recognition module 710 may determine a location of the endoscope 614 present within the body of the subject patient. The location of the endoscope may be determined using the ultrasound device 612, at step 814. Post determining the location of the endoscope 614, the measurement recognition module 710 may determine a distance of the endoscope 614 from the one or more internal organs of the subject patient, at step 816. The one or more internal organs may be viewed by the one or more cameras 616 of the endoscope 614.

Successive to determining, the measurement recognition module 710 may display the location of the endoscope 614 and the distance of the endoscope 614 from the one or more internal organs, at step 818. In one case, the location of the endoscope 614 and the distance of the endoscope 614 may be displayed to the surgeon using the AR display 610. In one case, the distance of the endoscope 614 from the one or more internal organs may be displayed using a sub-window matrix. For example, an “XYZ” location of the endoscope 614, and the distance between the “XYZ” location and the one or more internal organs may be displayed, using the sub-window. It should be noted that the distance may be present a minimum 3D distance, or as a ΔX, ΔY, and ΔZ distances. In another example, the AR display 610 may display distances that are below a threshold distance to avoid overload of information. In one case, the AR display 610 may display the distance as a function of speed of the endoscope 614 (i.e., distance is 1 mm per second). During such case, the AR display 610 may display a distance projection vector i.e., a vector line shown in a direction of travel of the endoscope 614, with a distance ruler set on the vector line.

Thereafter, the measurement recognition module 710 may store the recording m the electronic medical procedure database 606, at step 820.

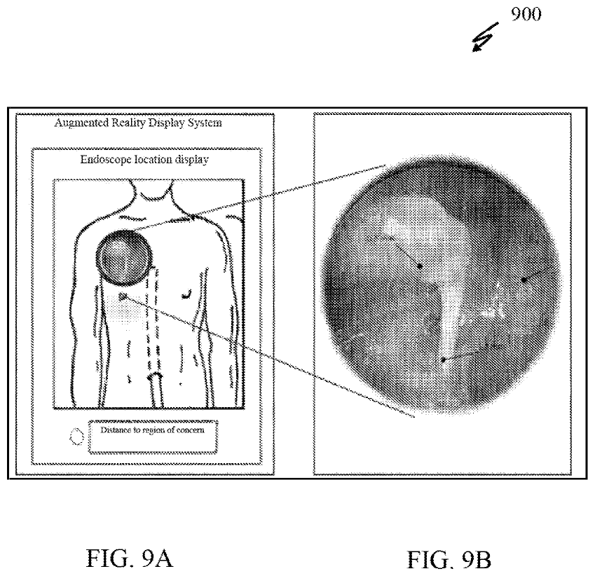

FIGS. 9A and 9B collectively illustrate a Graphical User Interface (GUI) of the user device 608, presenting information related to the surgical procedure, according to an embodiment. FIGS. 9A and 9B comprise the GUI, of the user device 608, explained in conjunction with the elements disclosed in Figures explained above.

The surgeon may track the surgical procedure either using the AR display 610 or the GUI of the user device 608. The GUI of the user device 608 may allow the surgeon to perform various functions such as, but not limited to, selecting the region of interest, creating the distance based rules, viewing information related to the point of view, the real-time cameras 620, viewing the AR images, and viewing the distance of the endoscope 614 from the one or more internal organs.

As shown in FIG. 9A, the surgeon may view the subject patient and the location of the endoscope 614 using the AR display 610. At a bottom of the AR display 610, the distance of the endoscope 614 to the region of interest may be displayed. It should be noted that the distance of the endoscope 614 to the region of interest may be determined based on a location of the region of interest and the location of the endoscope 614. For example, the distance to the region of interest is 5:6:4 mm, as shown in FIG. 9A.

As shown in FIG. 9B, the point of view of the endoscope 614 captured by the one or more cameras 616 of the endoscope 614, may be displayed on the GUI of the user device 608. Also, the distance of the one or more internal organs from the endoscope 614 may be displayed. For example, as shown in FIG. 9B, a distance to a first internal organ is 0.5 mm, a distance to a second internal organ is 1.1 mm, and a distance to a third internal organ is 2.1 mm.

Thus, it is evident from the above description that the system and method mentioned above may allow the surgeon to control the endoscope 614 during the surgical procedure. The method displays to the surgeon, the point of view of the endoscope 614, the location of the endoscope 614 and the distance of the endoscope 614 from the one or more internal organs, and thus makes the system and method efficient for performing endoscopic surgeries.

In an illustrative embodiment, any of the operations, processes, etc. described herein can be implemented as computer-readable instructions stored on a computer-readable medium. The computer-readable instructions can be executed by a processor of a mobile unit, a network element, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific. Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes shows different components contained within, or connected with, different other components. It is to be understood that such depicted architectures are merely examples, and that in fact many other architectures can be implemented which achieve the same functionality, In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically rateable and/or physically interacting components and/or wirelessly interactable and/or wirelessly interacting components and/or logically interacting and/or logically interactable components.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed:

1. A system for robotically-guiding surgical drill, comprising:
a surgical drill, comprising:
a drill bit holder configured to receive a surgical drill bit for interacting with a target bone or tissue;
cameras, provided around a periphery of the drill bit holder, each camera configured to capture point-of-view (PoV) images of the target bone or tissue of a subject patient;
a display configured to:
produce a composite PoV image of the target bone or tissue of the subject patient by integrating the images captured by each of the cameras, and
display an augmented reality (AR) anatomical image or virtual reality (VR) anatomical image based on a viewing angle and a region proximate to the composite PoV image;
a hand-held guidance device configured to guide the surgical drill remotely based on the displayed AR or VR anatomical image,
wherein the display is further configured to update the displayed AR or VR anatomical image based on a change in the viewing angle and the area proximate to the composite PoV image.

2. The system according to claim 1, wherein the digital reader is configured to read a serial number on the surgical drill bit to identify the drill bit that is appropriate for interfacing with the target bone or tissue.

3. The system according to claim 1, wherein
the digital reader is a near field communication (NFC) reader, and
the surgical drill bit has an encoded NFC chip attached thereto.

4. The system according to claim 1, wherein the drill bit holder is configured to individually receive different drill bits.

5. The system according to claim 1, wherein the drill bit holder comprises:
a drill bit identifying module configured to identify parameters of the received surgical drill bit.

6. The system according to claim 1, wherein the drill bit holder comprises:
a drill bit identifying module configured to identify at least one of a type of the surgical drill, a type of the received surgical drill bit, a size of the received surgical drill bit, or an absolute position of the received surgical drill bit.

7. The system according to claim 1, wherein the absolute position of the received surgical drill bit is in an XYZ coordinate system referencing an operating table.

8. The system according to claim 1, wherein the hand-held guidance device is a same type of drill as the surgical drill.

9. The system according to claim 1, wherein the surgical drill further comprises at least one Light Amplification by Stimulated Emission of Radiation (LASER) device disposed around the drill bit holder.

10. The system according to claim 9, wherein the LASER device is configured to guide a user of the hand-held guidance device.

11. The system according to claim 10, wherein guidance of the surgical based on the displayed AR or VR anatomical image is at least partially manual.

12. The system according to claim 10, wherein guidance of the surgical based on the displayed AR or VR anatomical image is at least partially automatic.

13. The system according to claim 4, wherein at least one of the different drill bits is configured to cut soft tissue.

14. The system according to claim 13, wherein at least one of the different drill bits has teeth disposed on an inner-housing thereof.

15. The system according to claim 13, wherein at least one of the different drill bits has serrated edges on an internal cutting surface.

16. The system according to claim 13, wherein at least one of the different drill bits has serrated edges on an external cutting surface.

17. The system according to claim 13, wherein at least one of the different drill bits is configured to provide a polished edged to cut tissue.

18. The system according to claim 13, wherein at least one of the different drill bits is configured for use in a laparoscopic surgical procedure.

19. The system according to claim 13, wherein at least one of the different drill bits is configured for use in a spinal surgical procedure.

20. The system according to claim 4, wherein the drill bit holder includes an accelerometer to detect changes in position of the surgical drill.
